(19) United States
(12) Patent Application Publication
(10) Pub. No.: US 2026/0345076 A1
(43) Pub. Date: Nov. 5, 2026

(54) REPLACEABLE PATCH FOR WEARABLE TRAINING COMPUTER AND WEARABLE TRAINING COMPUTER

(71) Applicant: Polar Electro Oy, KEMPELE (FI)

(72) Inventors: Ville MAJAVA, KEMPELE (FI); Matti KINNUNEN, KEMPELE (FI)

(73) Assignee: Polar Electro Oy, KEMPELE (FI)

(21) Appl. No.: 19/114,439

(22) PCT Filed: Oct. 4, 2023
(86) PCT No.: PCT/FI2023/050569

§ 371 Date: Mar. 22, 2025

(30) Foreign Application Priority Data
Oct. 5, 2022 (GB) 2214630.2

(51) Int. Cl. A61B 5/00; A61B 5/0205; A61B 5/024; A61B 5/145; A61B 10/00

(52) U.S. Cl. CPC A61B 5/681; A61B 5/0205; A61B 5/14517; A61B 10/0064; A61B 5/02416; A61B 5/02438; A61B 5/14532; A61B 5/14546; A61B 2505/09; A61B 2560/0443

(57) ABSTRACT

A replaceable patch for a wearable training computer includes a plasmonic bioactive layer for a surface plasmonic resonance measurement, at least one optical window for a light of at least one photoplethysmogram sensor of the wearable training computer, and guiding means for guiding positioning of the replaceable patch on the wearable training computer such that the light of the at least one photoplethysmogram sensor can pass the at least one optical window. The wearable training computer includes a surface plasmonic sensor configured to measure biomolecular composition of a sample from a user of the wearable training computer, the photoplethysmogram sensor configured to measure cardiac activity of the user of the wearable training computer, and guiding means for guiding positioning of the replaceable patch on the wearable training computer such that the light of the photoplethysmogram sensor can pass at least one optical window of the replaceable patch.

### CROSS-REFERENCE TO RELATED APPLICATIONS

This application is a national phase application of International Application No. PCT/FI 2023/050569, filed Oct. 4, 2023, which claims benefit and priority to Great Britain Application No. 2214630.2, filed Oct. 5, 2022, which are incorporated by reference herein in their entireties.

### BACKGROUND

### Technical Field

The invention relates to a field of wearable training computers, especially a replaceable patch for the wearable training computers.

### SUMMARY

Surface plasmon resonance (SPR) phenomenon is a principle behind many biosensor applications. The SPR based sensors may be used for measuring many parameters from sweat of a user of a wearable training computer, for example. Applying of the SPR in the wearable training computer causes many structural challenges. The aim of the invention is to alleviate these challenges.

The present invention is defined by the subject matter of the independent claims.

Embodiments are defined in the dependent claims.

The embodiments and features, if any, described in this specification that do not fall under the scope of the independent claim are to be interpreted as examples useful for understanding various embodiments of the invention.

### BRIEF DESCRIPTION OF THE DRAWINGS

In the following the invention will be described in greater detail by means of preferred embodiments with reference to the attached drawings, in which

FIGS. 1 and 5B illustrate a wearable training computer with a replaceable patch according to embodiments of the invention;

FIGS. 2 and 3 illustrate the replaceable patch according to an embodiment of the invention;

FIG. 4 illustrates one specific feature of the replaceable patch according to an embodiment of the invention;

FIG. 5A illustrates the wearable training computer according to an embodiment of the invention; and

FIGS. 6A, 6B and 6C illustrate guiding and attaching means of the replaceable patch and the wearable training computer according to embodiments of the invention.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

Embodiments of the invention relate to a wearable training computer configured to carry out measurements during a physical exercise performed by a user. The wearable training computer may be a portable system attachable to the user's body. The wearable training computer is configured to measure physiological training data from the user's performance during the physical exercise and to output the training data to the user via a user interface of the training computer and/or via a user interface of another apparatus.

In an embodiment, the wearable training computer may further comprise an apparatus configured to be attached to the object. Such an apparatus may comprise an attachment structure designed and arranged to receive the training computer in a fixed, integrated, or detachable manner and to attach the training computer to the object. The attachment may be realized by a band that may be designed to encircle the object such that the band is attached around the object. The band may comprise locking parts at ends of the band where the locking parts form mutually counterparts such as a buckle and a catch. The locking parts may fix the band around the object as is commonly known in the field of wristwatches, wrist computers etc. Other forms of attachment of wearable devices are equally possible, e.g. the training computer may be integrated or attached to a garment such as a shirt, pants, harness, strap, or headwear.

Surface plasmon resonance (SPR) can be defined as a resonant oscillation of electrons stimulated by an incident light at an interface between two media, e.g. an interface between metal and dielectric material. The oscillation enables detection of changes in boundary conditions, and further detection of adsorption of biomolecules to the surface. Thus, surface plasmonic sensors may be applied for measuring adsorptions of material onto the surface of planar metal or metal nanoparticles. In the context of biosensors, the material may be a biochemical substance. The absorption changes optical properties of the interface (the refractive index) as a function of the proportion of the absorbed material and the compound of the absorbed material. Accordingly, properties of the material can be measured by measuring the change in the optical properties. This may be conducted by emitting light to the interface and measuring changes in the light reflected from the interface, e.g. changes in reflected light intensity and/or angle shift.

In a field of the wearable training computers, the surface plasmonic sensors may be used for measuring many parameters from sweat of the user. The parameters refer to biochemical substances like glucose for determining energy consumption and nutrition state of the user, lactate for determining intensity of an exercise performed by the user, and cortisol for determining a recovery state of the user, for example. A receptor agent may be applied on the metal surface for measuring the biochemical substances. The receptor agent may be a binding agent associating with the measured biochemical substance to amplify or enable the change in the optical properties. The receptor agent may be specific to the biochemical substance measured, e.g. the glucose, lactate, or cortisol. The respective receptor agent may be selected according to the state-of-the-art. For example, a biocompatible borate polymer, PAA-ran-PAAPBA, and alternatively glucose/galactose-binding protein have been used in connection with glucose SPR measurements. In cortisol SPR measurements, a cortisol antibody has been used as the receptor agent. In other words, a first receptor agent may be used for measuring a first biochemical substance and a second receptor agent may be used for measuring a second biochemical substance etc. Hence, there shall be a plurality of the different biochemical substances available when measuring a plurality of the biochemical substances.

Based on the above, applying of the surface plasmonic sensor(s) in the wearable training computer for measuring a plurality of the biochemical substances, sets many challenges to a structure. One such challenge is that conventionally the SPR measurements have been made in laboratory conditions and training environments cannot meet the laboratory conditions. Another problem is that once the receptor agent is mixed with the biochemical substance and the SPR measurements have been made, the measurement surface needs to be ‘cleaned’, i.e. the biochemical substance removed so that it does not contaminate later measurements. Yet another problem is that in the case of training computers, an ordinary user would need to be able to carry out the SPR measurements. Yet another problem comes from the already small size of the wearable training computers that incorporate various sensors such as a heart activity sensor, an oxygen consumption sensor, etc. And yet another problem is integration of various sensors in a small-sized training computer such as a wrist device. The solution of the invention is aimed to alleviate these challenges.

According to a first aspect, there is provided a replaceable patch for the wearable training computer, comprising a plasmonic bioactive layer for a surface plasmonic resonance measurement, at least one optical window for a light of at least one photoplethysmogram sensor of the wearable training computer, and guiding means for guiding positioning of the replaceable patch on the wearable training computer such that the light of the at least one photoplethysmogram sensor can pass the at least one optical window.

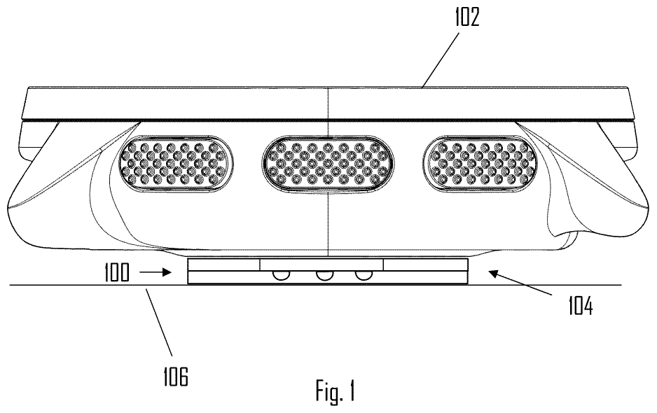

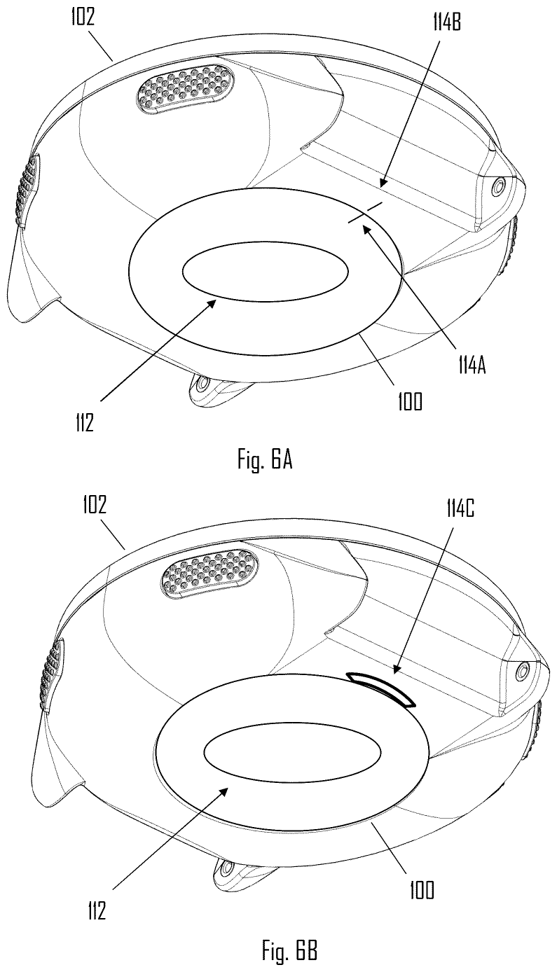

Referring to FIG. 1, the replaceable patch 100 (also the patch later in this application) is removably coupled with the wearable training computer 102 such that it stays in use (e.g. during physical exercises such as running, cycling, or gym exercises) between a bottom surface 104 of the wearable training computer 102 and a skin 106 of the user of the wearable training computer 102. The bottom surface 104 refers to a part of the training computer in which the sensors like the photoplethysmogram (PPG) sensor and the SPR sensor are placed as illustrated in FIG. 5, for example. This part is against the skin of the user for example when the training computer is in a wrist of the user. The user of the training computer may install the patch to the wearable training computer when he/she want to use the surface plasmonic resonance measurement features. The patch is replaceable allowing change of the patch when needed. The patch is configured to be used for a determined number of times (measurement occasions) and, when the patch cannot be used for the surface plasmonic resonance measurement anymore, it can be removed and replaced by a new patch. The number of measurement occasions may be defined by the capacity of the plasmonic bioactive layer to carry out the SPR measurements. As described above, the bioactive nature may be based on the receptor agent of the plasmonic bioactive layer, and the receptor agent may be consumed in the SPR measurements. The wearable training computer illustrated in Figures is a wrist device, without a wrist strap, but the embodiments described herein are directly applicable to the other wearable training computer or more generally to wearable sensors listed above.

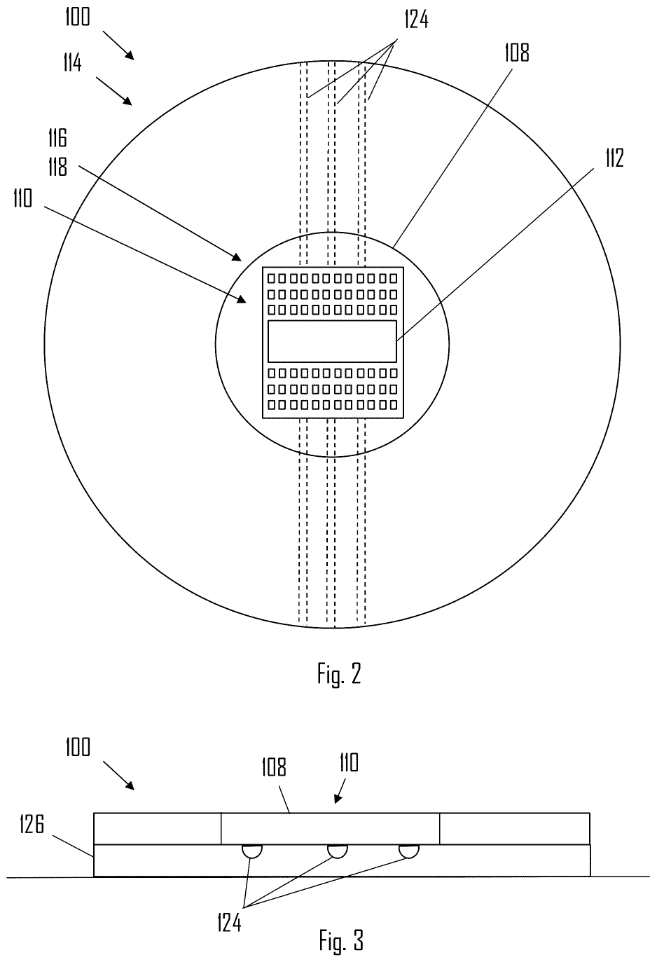

Referring to FIG. 2 which illustrates the replaceable patch 100 from a top view. The replaceable patch 100 comprises the plasmonic bioactive layer 108 configured to enable the surface plasmon resonance measurement with the wearable training computer 102. The wearable training computer may comprise a surface plasmonic sensor head configured to be used for the surface plasmon resonance measurement.

In an embodiment, the plasmonic bioactive layer comprises a sensing layer. The sensing layer may be made of metal, in other words, the sensing layer may be a metallic layer placed in the plasmonic bioactive layer of the patch. In the preferred embodiment, the sensing layer is made of gold. Other metals may be used as well instead of gold like silver, copper, titanium and/or chromium, for example. The material may also be combination of two or more metals. The receptor agent may be applied on the metallic layer (sensing layer) when performing the SPR measurements.

Still referring to FIG. 2, the plasmonic bioactive layer 108 further comprises one or more plasmonic sensor elements 110 (biochemical receptors) configured to be activated electrically. The plasmonic sensor elements may comprise the above-described receptor agent used for the surface plasmon resonance measurement. As described above, the receptor agent may be used for measuring the biochemical substances from sweat of the user of the wearable training computer. The sensor element may be covered with a thin hermetic layer such that the receptor agent is inside the hermetic layer. The hermetic layer may be removed with light, acoustic or electric energy, for example. Removing the hermetic layer releases the receptor agent. In one embodiment, a thin metallic layer is used to cover the sensor element which can be removed by electric current via electrolysis between a body or a chassis of the wearable training computer when sweat is working as electrolyte, for example. As described, the activation of the plasmonic sensor element may refer to removal or braking of the hermetic layer and releasing the receptor agent for measuring. The receptor agent may be released into a reservoir for the SPR measurements, and the sweat may be directed to the same reservoir, e.g. by using fluidic channel(s) described herein.

As described above, the plasmonic bioactive layer 108 may comprise a plurality of the sensor elements 110. In an embodiment, each sensor element in the plasmonic bioactive layer can be activated separately. Then one patch having a plurality of the sensor elements can be used multiple times for the SPR measurements. If there are 8 sensor elements in the plasmonic bioactive layer, the patch may be used 8 times for the SPR measurements, for example. After this the patch shall be replaced.

Some biochemical substances associate with the sensing layer spontaneously and directly without the need for the receptor agent to amplify the SPR characteristic. For example, the literature demonstrates scenarios where the glucose is measured directly without the receptor agent. Therefore, in an embodiment the plasmonic bioactive layer comprises the sensing layer without the plasmonic sensor elements and consequently without the receptor agent.

The surface plasmonic sensor (head) may comprise a light source, a lightguide element such as a prism or a diffractive optical element, and a detector. As known in the art of SPR sensors, the SPR sensor head using the prism may be configured to measure reflection of light at the metal film while the SPR sensor head using the diffractive optical element may be configured to measure diffraction of the light at the metal film. Both lightguide elements are suitable for measuring the above-described optical changes caused by the absorption of the biochemical substance and the receptor agent on the sensing layer. The diffractive optical element may be preferable with the wearable training computer because of the smaller size in depth direction. Additionally, with a planar optical lightguide element provides for more reliable focusing of the light to a desired part of the sensing layer. The detector may be a charge-coupled device camera (CCD—camera) or an array detector, for example. In an embodiment, the plasmonic sensor head in the training computer is configured to cover all the plasmonic sensor elements of the patch. In other words, the plasmonic sensor elements are in an operating range of the plasmonic sensor head(s). In this embodiment, the plasmonic sensor head may comprise optics for focusing the light of the sensor to an appropriate plasmonic sensor element selected for the particular measurement. In another embodiment, the training computer comprises a plurality of the plasmonic sensor heads wherein a first sensor head covers a first group of the plasmonic sensor elements, and a second sensor head covers a second group of the plasmonic sensor elements etc. Hence, the elements may be divided groups and there is a sensor head for each group. Then, focusing of the sensor head to the sensor elements may not be needed.

In an embodiment, the plasmonic sensor element is configured to be activated by an electric input from the wearable training computer. As described above, there may be a plurality of the sensor elements in the plasmonic bioactive layer, and each sensor element may be activated separately. Each sensor element may be activated by the electrical input from the wearable training computer. In an embodiment, the wearable training computer is configured to determine which sensor element shall be activated. In an embodiment where the SPR optics comprises an adjustable focusing element, the wearable training computer is further configured to align the SPR sensor head (the SPR optics) with the activated sensor element such that the SPR measurement is possible to be performed. Hence, the wearable training computer knows which plasmonic sensor element is activated and, as a consequence, is configured to focus the SPR sensor head to the activated sensor element.

The correct positioning of the patch is very essential to enable aligning of the SPR sensor head with the activated sensor element. Therefore, the guiding means is very essential feature of the invention. For example, when a first patch is coupled with the training computer, the training computer may activate a first sensor element, and then focus the SPR sensor head to the first sensor element. This enables a first SPR measurement with the first patch. Next time, the wearable training computer may activate a second sensor element, different from the first sensor element, and then focus the SPR sensor head to the second sensor element to enable a second SPR measurement with the first patch etc. When all the sensor elements are used, the first patch is removed from the training computer and a second patch is attached on the training computer.

In an embodiment, the wearable training computer is configured to detect when all the sensor elements are used, and it is time to change the patch. Upon so detecting, the training computer may inform the user via a user interface that it is time to change the patch. The training computer may further inform the user how many measurements can be performed (is still left) with the attached patch. Then the user knows beforehand when the patch shall be changed. In embodiments where there exists only one type of patch with a default number of plasmonic sensor elements, the number of plasmonic sensor elements and their respective locations on the patch may have been hardcoded into the wearable training computer. However, the number and/or positioning of the plasmonic sensor elements may be variable in a case where multiple different patches are available. In such a case, the replaceable patch may comprise an identifier readable by the wearable training computer, the identifier indicating the type of the replaceable patch. The wearable training computer may store a database storing different types and respective numbers and locations of the plasmonic sensor elements for each of the different types. Accordingly, the wearable training computer may support patches with different arrangements of the plasmonic sensor elements. The identifier may be provided in the form of a memory circuit readable by the wearable training computer via wireless or wired transfer. Since there is a wired coupling for the purpose of activating the plasmonic sensor elements, there may be a wired coupling between the memory circuit and the wearable training computer for the purpose of reading the identifier. An example of the wireless transfer is near-field communications (NFC) or another method based on radio frequency identification (RFID) technology. Yet another embodiment is the reception of the identifier from the user via a user interface of the wearable training computer.

In an embodiment, the guiding means are configured to guide positioning of the patch on the training computer such that the plasmonic sensor head(s) becomes aligned with the plasmonic sensor elements and thus enabling the conduction of the SPR measurements.

In an embodiment, the sensor may comprise a micromirror for focusing the light of the sensor to the plasmonic sensor element selected/activated for the measurement. The micromirror or a micromirror array may be placed between the light source and the prism or the diffractive element to focus the light to the right spot in plasmonic sensor elements, for example. This embodiment may be used if adaptive focusing of the sensor to the sensor elements is needed. As known in the art of micromirrors, such a micromirror (array) may be realized as a microelectromechanical system (MEMS) device.

Still referring to FIG. 2, the replaceable patch 100 further comprises the at least one optical window 112 for light of at least one photoplethysmogram (PPG) sensor of the wearable training computer 102. A shape of the optical window may vary according to the needs. The size and locations of the PPG-sensor elements (photo emitter(s) and detector(s)) 120 at the bottom surface 104 of the training computer 102 may affect the size and shape of the optical window 112, as illustrated in FIG. 5B. In other words, the shape of the optical window may be based on the location of other sensors of the training computer such that light of the other sensors can pass the optical window. As illustrated in FIG. 5B, the PPG-sensors 120 may be placed on the bottom surface 104 such that they form a cross shaped pattern, and the shape of the optical window 112 is configured to match to this pattern. In other words, the optical window may be the cross shaped as well. A technical effect of the optical window is that the patch disposed on the PPG sensor head will not degrade the performance of the PPG measurements and the PPG measurements may be conducted with high signal quality. The PPG-sensor is used for measuring cardiac activity of the user of the wearable training computer. The light of the PPG-sensor shall have access through the patch to the skin of the user via the optical window. The optical window is configured to enable access of the light through the patch and enable function of the PPG-sensor. The optical window may refer any kind of window (opening in a structure of the patch) which allows the light of the PPG-sensor to pass it. The optical window may be, at least partly, transparent to the light of the PPG sensor, for example. The optical window may be placed in the plasmonic bioactive layer of the patch, for example. The light of the PPG-sensor may not pass properly the structure of the patch without the optical window, therefore the optical window is needed to ensure proper function of the PPG sensors. The optical window may be a hole in the plasmonic bioactive layer. Alternatively, material of the patch may form the optical window such that the window has the above-described properties. The material of the optical window may be fully transparent to light, or it may be frequency-selective such that it is transparent to the wavelengths of the PPG sensor while it blocks other wavelengths of the light, thus reducing ambient light noise, for example.

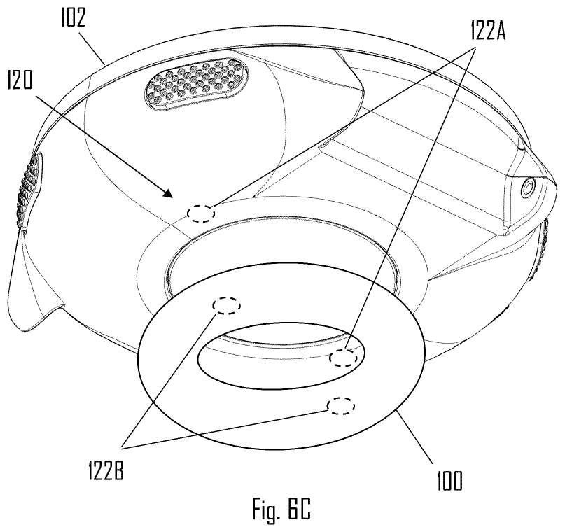

Referring to FIGS. 6A and 6B, the replaceable patch 100 further comprises the guiding means 114 for guiding positioning of the replaceable patch 100 on the wearable training computer 102 such that the light of the at least one photoplethysmogram sensor can pass the at least one optical window 112 and to enable the SPR measurements. Guiding of the replaceable patch to the right position on the wearable training computer is very essential since the wrong position can block the light of the PPG-sensor and prevent measuring of the cardiac activity of the user, or at least degrade the performance of the PPG measurements. Similarly, misalignment of the SPR sensor head with the correct plasmonic sensor element(s) degrades or prevents the performance of the SPR measurements. As described above, the patch may be installed at the bottom surface of the wearable training computer where the sensors are configured to interact with the user's skin. The guiding means are configured to help the user to set the patch to the right position on the bottom surface of the wearable training computer. In the right position, the PPG-sensor(s) and the optical window are aligned allowing normal use of the PPG-sensor and measuring of the cardiac activity when the patch is attached to the training computer. Similarly, the guiding means enable alignment of the plasmonic sensor elements and the SPR measurement head. The alignment may be understood such that the guiding means arrange the plasmonic sensor element(s) of the patch on the SPR measurement head and the optical window on the PPG sensor head.

The guiding means are an important feature of the invention. The whole functioning of the patch is based on the correct position of the patch on the training computer. The guiding means are configured to enable attaching of the patch correctly on the training computer also in a challenging environment like in outdoor conditions and/or during the physical exercises, for example.

A shape of the patch may vary according to the needs. The shape may be configured to match a shape of the bottom surface of the wearable training computer. The shape of the bottom surface of the training computer may vary between different models of the wearable training computer which may affect the shape of the patch. In other words, the certain training computer model may have the certain patch configured to fit the shape of the model. Different models may be configured to employ different embodiments of the patch, e.g. different guiding means. The shape of the patch may be substantially round, for example. In other embodiments, the shape of the patch is asymmetrical to facilitate the correct positioning. In such a case, the asymmetric shape together with a counterpart shape at the bottom of the wearable training computer operate as the guiding means.

The replaceable patch enables applying the surface plasmon resonance measurements to the wearable training computer. Using the patch is effortless and easy. The user can attach the patch on the training computer when he/she wants to perform the biochemical-substance-based measurements. The other functions of the wearable training computer can be used at the same time, like the PPG-sensors for measuring the cardiac activity. In other words, the patch enables performing both the SPR and PPG measurements during a physical exercise such as a running or cycling exercise. The guiding means ensures that the patch is easy to install in the right place and does not require a professional. A life cycle of the singe patch may be limited which means that the one patch may be used only for certain times for the measuring. When the end of the life cycle of the patch is achieved, the user can remove the used patch from the training computer and replace it by a new one.

In an embodiment, the plasmonic bioactive layer 108 comprises an array 116 of plasmonic sensor elements 110 configured to be activated by an electric signal from the wearable training computer. The plasmonic sensor elements of the array may be activated separately, which enables using the patch multiple times. For example, the array may comprise six plasmonic sensor elements comprising respective receptor agents to associate with a determined biochemical substance for the SPR measurements, which means that the patch may be used six times for measuring the biochemical substance from sweat of the user of the wearable training computer. Obviously, six is only an example, and other (plural) number of uses may be selected. The number of the plasmonic sensor elements in the array may vary according to the needs.

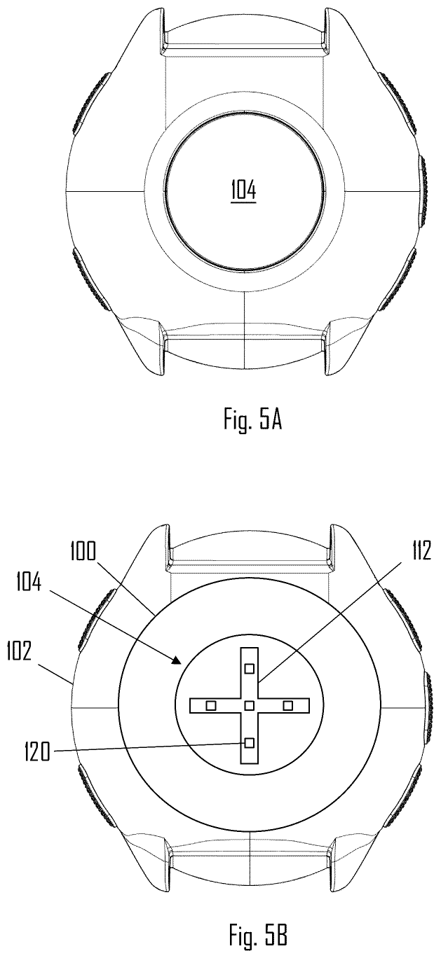

Referring now to FIG. 4, in an embodiment, the array 116 of the electrically active plasmonic sensor elements 110 comprise at least one subset SS1, SS2, SS3 with a first receptor agent for measuring a first biochemical substance. As described above, the receptor agent is used when measuring the biochemical substance from sweat of the user. The different biochemical substances may require the different antibodies, in other words, one receptor agent may be suitable for measuring one biochemical substance. The subset may comprise a plurality of the plasmonic sensor elements with the receptor agent. The plasmonic sensor elements in the array may comprise the first subset of the first receptor agent configured to be used for measuring the first biochemical substance. Hence, the first subset with the first receptor agent may be used multiple times for measuring the first biochemical substance.

Still referring to FIG. 4, in an embodiment, the array 114 of the plasmonic sensor elements 110 configured to be activated by the electrical input (stimulus) from the training computer comprises a plurality of the subsets SS1, SS2, SS3 with a plurality of the antibodies for measuring a plurality of the biochemical substances. For example, the first subset SS1 may comprise the first receptor agent for measuring the first biochemical substance (e.g. the glucose), the second subset SS2 may comprise the second receptor agent for measuring the second biochemical substance (e.g. the lactate), and third subset SS3 may comprise the third receptor agent for measuring the third biochemical substance (e.g. the cortisol). Each subset may comprise multiple plasmonic sensor elements, or only some of the subsets may comprise multiple plasmonic sensor elements. For example, the first subset SS1 may comprise eight plasmonic sensor elements 110 allowing eight measurements times of the first biochemical substance as illustrated in FIG. 4. The number of plasmonic sensor elements in each subset may be selected independently. The same applies to further subset(s) of plasmonic sensor elements configured to measure one or more further biochemical substances, if any. The number of plasmonic sensor elements per subset may be selected on the basis of estimated frequency of each measurement. For example, if it is anticipated that one biochemical substance shall be measured more frequently than another, the number of plasmonic sensor elements containing receptor agent for that biochemical substance may be greater than the number the number of plasmonic sensor elements containing receptor agent for the other biochemical substance. In other words, the number of plasmonic sensor elements per subset may be different for different subset.

In an embodiment, the biochemical substance to be measured from sweat of the user of the wearable training computer comprises at least glucose, lactate and/or cortisol. Still referring to FIG. 4, the first subset SS1 may comprise the first receptor agent for measuring the glucose, the second subset SS2 may comprise the second receptor agent for measuring the lactate, and the third subset SS3 may comprise the third receptor agent for measuring the cortisol, for example. In the field of the training computers, the glucose can be used for determining energy consumption and nutrition state of the user, the lactate can be used for determining intensity of an exercise performed by the user and cortisol can be used for determining a recovery state of the user. Hence, the replaceable patch enables measuring of the parameters that may not normally be possible with the wearable training computer in a daily use.

Let's now look at FIG. 2, in an embodiment, the replaceable patch 100 further comprises at least one pilocarpine element 118 for inducing sweat generation of the user of the wearable training computer 102. As described above for the plasmonic sensor element(s), the pilocarpine element may be configured to be activated by an electric stimulus from the training computer. The pilocarpine element(s) 118 may be placed in the plasmonic bioactive layer 108 of the patch 100. The pilocarpine element may comprise pilocarpine nitrate that is known in the art as a cholinergic agent that binds to muscarinic receptors of eccrine sweat glands and induces sweat production. As described above, the biochemical substances are measured from sweat of the user and, therefore, excretion of sweat by the user is essential for the successful measurement. The pilocarpine element is configured to induce sweat generation of the user such that the desired measurement can be performed from sweat. The pilocarpine element may be activated electrically like the plasmonic sensor element. Gel discs may be used for storing the pilocarpine and an iontophoretic current passing through these discs may be used for delivering the pilocarpine into sweat glands which stimulates sweat production.

In an embodiment, the wearable training computer 102 is configured to determine activation of the electrically active plasmonic sensor elements 110 and/or the at least one electrically active pilocarpine element 118. The wearable training computer may comprise a processing circuitry configured to determinate activation of the plasmonic sensor element and/or the pilocarpine element. The user of the training computer may select via a user interface that he/she wants to measure some biochemical substance. Based on the selection of the user, the processing circuitry is configured to select and activate a plasmonic sensor element for the desired measurement by outputting an electric signal releasing the receptor agent in the selected plasmonic sensor element for the measurements. Further, the processing circuitry is configured to determine whether or not to activate the pilocarpine element and, upon determining to activate, conduct the activation by outputting an electric signal releasing the pilocarpine to the user's skin. In an embodiment, the pilocarpine element may be activated automatically when using (activating) the plasmonic sensor element. In another embodiment, the processing circuitry is configured to determine whether or not the activation of the pilocarpine element needed, e.g. by determining whether or not the user excretes sweat enough for measuring. The determination of the degree of sweating may be carried out through measurements conducted by the wearable training computer. For example, the degree of sweating can be measured from a galvanic skin response: the skin conductance increases in proportion to the degree of sweating. Measuring of a galvanic skin response (e.g. the skin conductance or equivalently skin resistance) may be used to determine presence of sweat on the skin of the user. If the processing circuitry determines that there is not sweat enough for measuring, e.g. the skin conductance is below a determined threshold, the pilocarpine element may be activated by the processing circuitry by outputting the electric signal to the pilocarpine element.

In an embodiment, the guiding means 114 comprises at least optical guiding means. The optical guiding means refer to any kind of a visual feature in the patch and/or training computer suitable for aligning the patch with the training computer. The optical guiding means may comprise a positioning mark in the patch and/or the training computer. For example, the patch 100 may have a first mark 114A which is configured to be aligned with a second mark 114B placed on the bottom surface 104 of the training computer 102 as illustrated in FIG. 6A. The positioning mark may refer to the mark(s) which is added especially for positioning the patch on the training computer. Still any other feature (like shapes) of the patch or the training computer which main purpose is not positioning may be used for positioning. In another embodiment, the patch may comprise a notch on an outer edge and the bottom surface of the training computer may comprise the positioning mark configured to be aligned with the notch. When the mark of the training computer is inside the notch, the patch is correctly in place. This embodiment is not illustrated in Figures. The patch and/or the training computer may comprise a plurality of the marks. For example, there may be three marks in the patch and the training computer which must be aligned to ensure the correct position of the patch in relation to the training computer.

In an embodiment, the optical window may be used for guiding the patch to the training computer. For example, the optical window 112 may be aligned with the sensors 120 placed at the bottom surface 104 of the training computer 102 as illustrated in FIG. 5B. As described above, the shape of the optical window may vary, and the shape may be configured to be aligned with the sensors enabling positioning of the patch to the bottom surface of the training computer.

In an embodiment, the guiding means 114 comprises the mechanical guiding means. The mechanical guiding means refer to the mechanical feature in the patch and/or in the training computer. Referring now to FIG. 6B, the bottom surface 104 of the training computer 102 may comprise one or more protrusions 114C configured to receive the edge of the patch 100. The protrusion may extend substantially perpendicularly from the bottom surface of the training computer. The protrusion is like a low wall in the bottom surface of the training computer. The protrusion may have the same shape as the edge. If the shape of the patch is round, the shape of the protrusion may be curved, for example. Then the edge of the patch can easily be set against the protrusion.

In an embodiment, the guiding means in the patch and/or the training computer comprise the optical guiding means like the positioning mark(s) 114A, 114B and further the mechanical guiding means like protrusion(s) 114C.

In an embodiment, the processing circuitry of the training computer is configured to determine whether or not the patch has been positioned correctly. The processing circuitry may determine can all the sensors at the bottom of the training computer be used normally when the patch is attached. The training computer may further inform the user is the patch positioned correctly. The processing circuitry may be configured to provide positioning instructions for the user via user interface of the training computer. For example, the training computer may determine that the patch is aside from the correct position and provide instructions for correcting the position. The determination may be based on a signal level of an optical signal received at a photo detector of the PPG sensor and/or the SPR sensor.

Referring to FIG. 6C, in an embodiment, the replaceable patch 100 further comprises attaching means 122 for removably attaching the replaceable patch 100 on the wearable training computer 102. The attaching means are configured to keep the patch attached in the training computer firmly but also enabling removal of the patch from the training computer. It is essential that the attaching means are firm enough such that the patch stays on the bottom surface of the training computer during the physical exercises. On the other hand, the attaching means shall enable easy removal of the patch from the training computer since the one patch may be used only limited times.

In an embodiment, the attaching means comprises a mechanical attaching. The mechanical attaching may comprise a locking mechanism configured openable to lock the patch on the training computer. Referring to FIG. 6C, in an embodiment, the mechanical locking 122 comprises one or more magnets. For example, the training computer 102 may comprise one or more magnets 122A and the patch 100 may comprise magnetic material 122B for forming the mechanical attach. It may also be the other way around such that the patch comprises one or more magnets and the training computer the magnetic material. In addition, both the patch and the training computer may comprise one or more magnets. The magnetic material refers to materials that can be attracted by the magnet like ferromagnetic materials, for example.

The magnet may be a permanent magnet and/or an electromagnet. The electromagnet can be activated and deactivated. For example, electromagnet may be activated when attaching the patch on the training computer and deactivated when removing the patch. The training computer may be configured to activate and deactivate the electromagnet.

In another embodiment, the attaching means comprises a chemical attaching. The chemical attaching may comprise an adhesive, for example. The patch may comprise a sticker part with the adhesive configured to be removably attached to the bottom surface of the training computer.

In an embodiment, the attaching means may be combination of the mechanical and the chemical attaching means.

In an embodiment, the guiding means comprises the attaching means. For example, the magnet(s) may also force the patch to the correct position in relation to the training computer. So, the magnet may be the attaching means as well as the guiding means.

In an embodiment, the replaceable patch is configured to cover, when coupled with the wearable training computer, substantially a whole bottom surface of the wearable training computer. A large area (size) of the patch enables firm attaching of the patch especially if the adhesive is used as the attaching means.

In an embodiment, the replaceable patch is a non-rigid film.

In an embodiment, the replaceable patch further comprises at least one opening for an electric coupling between the wearable training computer and a skin of the user of the wearable training computer. The opening refers to through hole such that a physical contact between the senor(s) of the wearable training computer and the skin is possible. The physical contact may be used for measuring an electrocardiogram (ECG) of the user, for example. The opening may be placed in the plasmonic bioactive layer 108 of the patch 100, for example. In an embodiment, the optical window comprises the at least one opening.

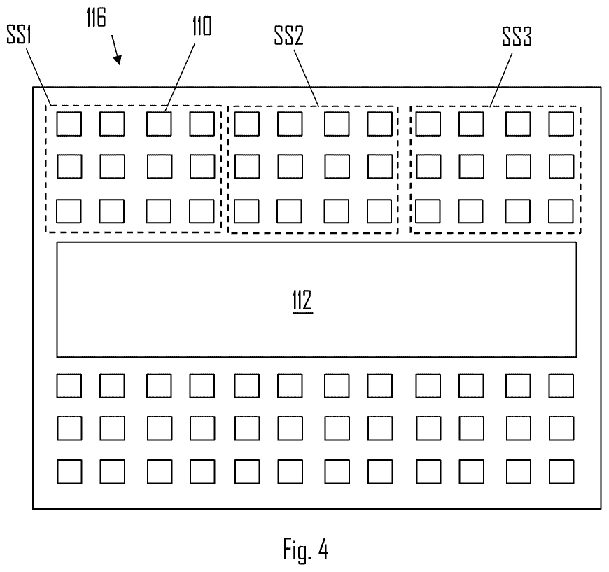

In an embodiment, the replaceable patch further comprises at least one fluidic channel for guiding sweat of the user of the wearable training computer to the plasmonic bioactive layer. The fluidic channel(s) may form a microfluidic network of microfluidic channel(s). Microfluidics may be understood to refer to control of fluid (sweat in the present context) geometrically constrained to a small scale, typically sub-millimetre, where surface forces dominate volumetric forces. Referring to FIGS. 2 and 3, the fluid channels 124 are configured to collect sweat from a large area and guiding sweat to the (activated) bioactive layer 108 such that sweat comes into contact with the plasmonic sensor element(s) 110 and receptor agent inside the element(s) 110. Referring now to FIG. 3 which is a cross section of the patch, the patch 100 may comprise a microfluidic layer 126 which is set against the skin 106 of the user of the training computer 102. The microfluidic layer 126 stays in use between the skin 106 and plasmonic bioactive layer 108. The layer 126 may comprise a network of the fluidic channels 124 configured to guide sweat to the plasmonic sensor elements 110. The fluidic channels capillary forces sweat to move towards the bioactive layer where the plasmonic sensor element are. The fluidic channels may guide the sweat to all plasmonic sensor elements but only the electrically activated plasmonic sensor element is enabled to react with the sweat. Meanwhile, the measurement head of the SPR sensor is directed to the activated plasmonic sensor element to measure the reaction optically.

In an embodiment combining the fluidic channel(s) and the array of plasmonic sensor elements, the fluidic channel(s) may funnel the sweat to all plasmonic sensor elements, not only the activated sensor element. Therefore, the sweat may contaminate the fluidic channel(s) and the inactive plasmonic sensor elements and degrade the accuracy of future SPR measurements. For that purpose, the processing circuitry of the wearable training computer may instruct the user to clean the exposed part of the replaceable patch before the next SPR measurements. The trigger for the cleaning may be after a certain number (one or more than one) of conducted SPR measurements. This may be particularly preferable when the number of plasmonic sensor elements in the replaceable patch is high, e.g. more than four or more than six or more than ten.

According to a second aspect of the invention, there is provided the wearable training computer, comprising a surface plasmonic sensor configured to measure biomolecular composition of a sample from the user of the wearable training computer, the photoplethysmogram sensor configured to measure cardiac activity of the user of the wearable training computer, and guiding means for guiding positioning of the replaceable patch on the wearable training computer such that light of the photoplethysmogram sensor can pass at least one optical window of the replaceable patch.

The wearable training computer may further comprise the above-described processing circuitry comprising at least one processor and at least one memory configured to control the operation of the wearable training computer and the measurements performed on the user. The processing circuitry may control the execution of both SPR and PPG measurements and to process measurement data acquired as a result of the measurements. With respect to the PPG measurements, the processing circuitry may carry out state-of-the art PPG processing functions to measure the user's cardiac activity including, for example, heart rate and its derivatives such as heart rate variability, or pulse transit time via PPG. The processing circuitry may control the conduction of the PPG measurements during not only the physical exercises but also between the exercises, e.g. during the user's sleep. With respect to the SPR measurements, the processing circuitry may trigger the SPR measurements under various conditions. The conditions may be different for different types of SPR measurements, particularly for measurements for different biochemical substances.

In an embodiment, the processing circuitry triggers the lactate measurements at the end of a high-intensity physical exercise such as high-intensity running or interval training exercise. The high intensity may be determined in terms of the heart rate or the classification of the exercise. The high intensity may refer to at least a certain amount of the physical activity during the exercise spent at the highest heart rate zone (indicating maximal training) or above the anaerobic threshold. Accordingly, the SPR measurements may be used to measure the amount of lactates in the user's blood and then compute a training load parameter and/or a recovery estimate parameters on the basis of the SPR measurements. As known in the art, the training load is analogous to strain of the exercise on the user's body, cardiovascular system, and/or muscles.

In an embodiment, the processing circuitry triggers the cortisol measurements upon detecting that the user has entered primary sleep or that the user woken up from the primary sleep. As known in the art, cortisol greatly affects the sleep quality and, as a consequence, the cortisol measurements may be used in connection with estimating the sleep quality of the user. A high cortisol level may be mapped with poor sleep quality while low cortisol levels may be mapped with good sleep quality. The cortisol measurements may further be used as an input to a recovery tracking estimation. Greater-than-normal cortisol level may be an indicator that the user has not yet fully recovered from strain of the past physical exercise(s).

In an embodiment, the processing circuitry triggers the glucose measurements during or at the end of the physical exercise in order to measure energy consumption or nutrition state. The glucose measurements may be used as a part of smart coaching system where at least some coaching instructions of the exercise are subject to the measurements. For example, the measurements may be triggered at the end of primary phases of the exercise in order to determine whether or not the user still has energy reserves to continue the exercise. Upon detecting that the user has not energy resources (the measured glucose levels are below a threshold), the processing circuitry may trigger a low-intensity cool-down phase ending the exercise. Upon that the user has energy resources (the measured glucose levels are above the threshold), the processing circuitry may trigger yet another workout phase. In another embodiment, the glucose measurements are a part of a nutrition coach algorithm of the wearable training computer. The nutrition coach algorithm may aim to measure the user's nutrition status via the SPR measurements (e.g. the glucose) and to provide nutritional instructions to meet at least one target for healthy nutrition. In such a case, the glucose measurements may be triggered by the processing circuitry upon receiving a user input via a user interface of the wearable training computer and, e.g. the user requesting for nutritional instructions in a nutrition coaching application executed in the wearable training computer. Upon measuring the user's glucose level, the processing circuitry may compute the nutritional instructions on the basis of the measurements. For example, if the glucose level is below a threshold, the processing circuitry may output an instruction to the user to eat something healthy that raises the glucose level and an amount to eat. The amount may be determined on the basis of the measured glucose level, e.g. whether the user is proposed to eat a snack (e.g. a fruit) or a main meal. The glucose measurements may even be a part of a smart coaching for a user suffering from diabetes, and it could help the user to maintain the blood sugar levels in a determined region.

In summary, the SPR measurements may belong to various functions of the wearable training computer, and the SPR measurements may be conducted simultaneously with the PPG measurements, and the measurement data from the SPR measurements may even be combined with measurement data from the PPG measurements, e.g. for the purpose of estimating the sleep quality or training load. Referring to FIG. 6A-6C , the wearable training computer 102 is configured to receive the replaceable patch 100. The training computer comprises the surface plasmonic sensor head configured to measure the biomolecular composition of the sample from the user of the wearable training computer. The biomolecular composition refers to the biochemical substance. As described above, the surface plasmonic sensor with the replaceable patch enables measuring of the biochemical substances from the user of the training computer. The wearable training computer further comprises the at least one PPG-sensor configured to measure cardiac activity of the user of the wearable training computer. The patch comprises the at least one optical window for enabling using of the PPG-sensors when the patch is attached to the training computer.

The wearable training computer further comprises the guiding means for guiding positioning of the replaceable patch on the bottom surface of the wearable training computer such that the light of the PPG-sensor(s) can pass the at least one optical window. As described above, the optical window is needed to ensure proper working of the PPG-sensors. The optical window shall be positioned correctly in the training computer for allowing access of the light of the sensor to the skin of the user. The guiding means in the training computer are configured to be used together with the guiding means of the replaceable patch for guiding the patch in the right position on the training computer. The features and functioning of the guiding means, presented in connection with the patch, are valid also for the training computer.

The wearable training computer may further comprise attaching means configured to enable attaching of the patch on the training computer. The features and functioning of the attaching means, presented in connection with the patch, are valid also for the training computer.

The invention provides a simple and efficient solution for applying the surface plasmonic resonance measurement in the wearable training computer. The invention enables measuring of the parameter from the user of the wearable training computer that normally may not be possible. The one patch may be used multiple times. The patch also enables measuring of the several different parameters from the user like glucose, lactate, and cortisol. The patch also allows normal use of the other functions of the training computer. For example, the cardiac activity measurement by the PPG-sensor(s) is possible when the patch is attached on the training computer. In addition, the patch is easy to install and remove. Guiding means help the user to attach the patch correctly on the training computer. The attaching means ensures that the patch stay firmly on the training computer during the physical exercises, but also enables easy removal of the patch.

The SPR measurements may be conducted only occasionally. It may be that the user has no use for the SPR measurements for an extended period of time. In such a case, it may not be meaningful to keep the replaceable patch attached to the wearable training computer. For example, the sweat and other substances may clog the microfluidic network and contaminate the future SPR measurements. However, it may be desirable to protect the measurement heads from scratches. For that purpose, a kit of replaceable patches may be provided, the kit comprising the replaceable parch according to any one of the above-described embodiments and, further, another replaceable patch (a protective patch) comprising the above-described or another optical window for the PPG sensor but no plasmonic bioactive layer. The protective patch may be used during such periods of time when there is no need for the SPR measurements.

The embodiments are described above in connection with the SPR measurements. A similar replaceable patch is equally applicable to other measurement techniques that may exist in wearable training computers having the PPG measurement capability. An example of such another measurement technique is one based on an optical fluorescent reader configured to measure a biochemical substance from a sample acquired from the user, e.g. from the sweat. The measured biochemical substance may be the glucose, for example. Fluorescent glucose biosensors as such are readily known in the literature, and they are used to measure the concentration of glucose via a sensitive protein that represents the glucose concentration by means of fluorescence.

According to an aspect, the replaceable patch described above in connection with the SPR measurements can be generalized and defined as follows: a replaceable patch for a wearable training computer, comprising: a bioactive layer for optically measuring a biochemical substance from a user's skin; at least one optical window for a light of at least one photoplethysmogram sensor of the wearable training computer; and guiding means for guiding positioning of the replaceable patch on the wearable training computer such that the light of the at least one photoplethysmogram sensor can pass the at least one optical window.

In an embodiment, the plasmonic bioactive layer described in the embodiments above is replaced by a fluorescence layer comprising fluorophore molecules that interact with the glucose to induce the fluorescence that can be measured optically. The fluorophore molecules may be the same conventionally used for the glucose measurements. The same applies to other biochemical substances such as the lactate and cortisol. The literature discloses suitable dyes or fluorophores that enable the measurement of each desired biochemical substance. The optical measurement head may use the principles described above, with the exception that the prism or diffraction optics may not be necessary in connection with the fluorescence measurements. The optical measurement head may comprise a light emitter in the form of a light emitting diode (LED) or laser for sample illumination and a photo detector in the form of a photo diode or the CCD for fluorescent sensing or imaging the illuminated sample comprising the fluorophore and the biochemical substance to be measured. The light emitter may alternatively be called a fluorescence excitation light source in the context of fluorescence measurements. The wavelength(s) employed may be adapted to the visible or near-infrared range, for example, nevertheless to the range that is matched with the used fluorophore molecules of the fluorescence layer. In the context of fluorescence measurements, elimination of ambient light noise is important and, for that purpose, there may be an optical (spectral) filter at least at the photo detector side. A similar spectral filter may be provided at the light emitter side for further improved performance.

With respect to the other features of the replaceable patch adapted to measure the biochemical substance via fluorescence or another optical technology, the replaceable patch may be designed according to any one of the embodiments described above for the replaceable patch for the SPR measurements. Similarly, the wearable training computer employing the replaceable patch adapted to measure the biochemical substance via fluorescence or another optical technology may be designed according to any one of the embodiments described above for the replaceable patch for the SPR measurements. The only difference is the optical measurement head that is designed and configured according to the appropriate measurement technology. For example, the replaceable patch may still be designed to have the array of sensor elements that can be activated separately by the wearable training computer, each sensor element comprising the fluorophore or another receptor agent. In an embodiment, the sensor elements comprise a first subset of plasmonic sensor elements and a second subset of fluorophore sensor elements. In such a case, the wearable training computer may have separate optical measurement heads for the SPR measurements and the fluorescence measurements.

As used in this application, the term ‘circuitry’ refers to all of the following: (a) hardware-only circuit implementations, such as implementations in only analog and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), graphics processing units (GPUs), processors, controllers, micro-controllers, microprocessors, other electronic units designed to perform the functions described herein, or a combination thereof. For firmware or software, the implementation can be carried out through modules of at least one chipset (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

It will be obvious to a person skilled in the art that, as the technology advances, the inventive concept can be implemented in various ways. The invention and its embodiments are not limited to the examples described above but may vary within the scope of the claims.

What is claimed is:

1. A replaceable patch for a wearable training computer, the replaceable patch for the wearable training computer, comprising:
a plasmonic bioactive layer for a surface plasmonic resonance measurement;
at least one optical window for a light of at least one photoplethysmogram sensor of the wearable training computer; and
guiding means for guiding positioning of the replaceable patch on the wearable training computer such that the light of the at least one photoplethysmogram sensor can pass the at least one optical window.

2. The replaceable patch of claim 1, wherein the plasmonic bioactive layer comprises an array of plasmonic sensor elements, wherein each sensor element is configured to be separately activated by an electric input from the wearable training computer.

3. The replaceable patch of claim 2, wherein the guiding means are further configured to guide positioning of the replaceable batch on the wearable training computer such that a light of a surface plasmonic sensor head of the wearable training computer can be focused on the activated sensor element.

4. The replaceable patch of claim 2, wherein the array of the electrically active plasmonic sensor elements comprises at least one subset with a first receptor agent for measuring a first biochemical substance.

5. The replaceable patch of claims 2, wherein the array of the electrically active plasmonic sensor elements comprises a plurality of the subsets with a plurality of the receptor agents for measuring a plurality of the biochemical substances non-invasively from sweat.

6. The replaceable patch of claims 4, wherein the biochemical substance to be measured comprises at least one of glucose, lactate and cortisol.

7. The replaceable patch of claim 1, wherein the replaceable patch further comprises at least one pilocarpine element for inducing sweat generation of a user of the wearable training computer, the pilocarpine element is configured to be activated by an electric input from the wearable training computer.

8. The replaceable patch of claim 1, wherein the plasmonic bioactive layer comprises a metallic layer.

9. The replaceable patch of claim 1, wherein the guiding means comprises at least optical guiding means and/or mechanical guiding means.

10. The replaceable patch of claim 1, wherein the replaceable patch further comprises attaching means for removably attaching the replaceable patch on the wearable training computer, wherein the attaching means comprises a mechanical attaching and/or a chemical attaching.

11. The replaceable patch of claim 10, wherein the guiding means comprises the attaching means.

12. The replaceable patch of claim 1, wherein the replaceable patch is configured to cover, when coupled with the wearable training computer, substantially a whole bottom surface of the wearable training computer.

13. The replaceable patch of claim 1, wherein the replaceable patch further comprises at least one opening for an electric coupling between the wearable training computer and a skin of the user of the wearable training computer.

14. The replaceable patch of claim 1, wherein the replaceable patch further comprises at least one microfluidic channel for guiding sweat from the user's skin to the plasmonic bioactive layer.

15. A wearable training computer, the wearable training computer comprising:
a surface plasmonic sensor configured to measure biomolecular composition of a sample from a user of the wearable training computer;
a photoplethysmogram sensor configured to measure cardiac activity of the user of the wearable training computer; and
guiding means for guiding positioning of a replaceable patch on the wearable training computer such that a light of the photoplethysmogram sensor can pass at least one optical window of the replaceable patch.
